(19) United States
(12) Patent Application Publication
(10) Pub. No.: US 2025/0129007 A1
(43) Pub. Date: Apr. 24, 2025

(54) PROCESS FOR PREPARING P-HYDROXYCINNAMIC ACID

(71) Applicant: SPECIALTY OPERATIONS FRANCE, Lyon (FR)

(72) Inventors: Eric Muller, Lyon (FR); Valery Dambrin, Irigny (FR)

(73) Assignee: SPECIALTY OPERATIONS FRANCE, Lyon (FR)

(21) Appl. No.: 18/833,182

(22) PCT Filed: Jan. 24, 2023
(86) PCT No.: PCT/EP2023/051610

§ 371 Date: Jul. 25, 2024

(30) Foreign Application Priority Data
Jan. 25, 2022 (FR) FR2200617

(51) Int. Cl. C07C 51/38

(52) U.S. Cl. CPC C07C 59/52; C07C 51/353

(57) ABSTRACT

The present invention relates to a process for preparing para-hydroxycinnamic acid from para-hydroxybenzaldehydes by reaction with malonic acid in the presence of at least one of an amino acid and acetic acid. The process may include a condensation reaction between a para-hydroxybenzaldehyde compound with malonic acid in the presence of at least one of an amino acid and acetic acid.

### FIELD OF THE INVENTION

The present invention relates to a process for preparing para-hydroxycinnamic acid, in particular from para-hydroxybenzaldehydes.

### PRIOR ART

Para-hydroxycinnamic acids are used in a large number of fields and in particular as synthesis intermediates. They can in particular be used in the synthesis of active molecules, antioxidants, flavorings/aromas, resins or polymers.

One of the methods for producing these para-hydroxycinnamic acids consists in using a Knoevenagel reaction.

However, the use of this type of process is little used industrially because it requires the use of a large amount of a toxic solvent: pyridine or toluene; mention may in particular be made of U.S. Pat. No. 7,572,809, JP 2018123127 and CN 103242163. The use of these solvents poses toxicological problems: these solvents can have effects on human health and the environment. Alternatives were able to be proposed with the use of aniline or piperidine, which are a little less dangerous, but it is still desirable to reduce the use thereof.

Furthermore, the performance of the reaction is affected by secondary reactions linked to the decarboxylation of malonic acid or of para-hydroxycinnamic acid. To overcome this decarboxylation problem, it is sometimes necessary to add a large amount of malonic acid, such as in particular in CN 1027756 or in Peyrot et al. ACS Sustainable Chemistry & Engineering, 2019, 7 (10), pp. 9422-9427.

In order to be able to industrially implement a process for preparing para-hydroxycinnamic acid from para-hydroxybenzaldehyde, it is therefore desirable to be able to provide a process having an improved environmental footprint and performance. It is also desirable to provide an industrial process that reduces the risks to human health. The present invention is targeted at the efficient and selective manufacture of para-hydroxycinnamic acid, on an industrial scale, in an economically viable manner and under good safety conditions.

### BRIEF DESCRIPTION

The present invention relates to a process for preparing para-hydroxycinnamic acid of formula (I)

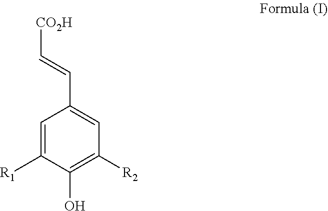

in which R1 and R2 are, identical or different, independently, chosen from the group consisting of hydrogen, linear or branched, optionally substituted, C1-C6 alkyl groups, linear or branched, optionally substituted, C1-C6 alkenyl groups, —CO₂R, —OR, in which R is chosen from hydrogen, linear or branched, optionally substituted, C1-C6 alkyl groups, linear or branched, optionally substituted, C1-C6 alkenyl groups,
in which a para-hydroxybenzaldehyde of formula (II) in which R1 and R2 are as described above

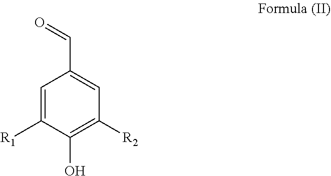

is reacted with malonic acid in the presence of at least one amino acid and acetic acid.

### DETAILED DESCRIPTION

In the context of the present invention, and unless otherwise indicated, the expression “between . . . and . . . ” includes the limits. Unless otherwise indicated, the percentages and ppm are percentages and ppm by mass.

In the context of the present invention, the expression “carbon of biobased origin” or “biobased carbon” refers to carbons of renewable origin such as live agricultural, plant, animal, fungal, microorganism, marine or forestry carbons in a natural environment in equilibrium with the atmosphere. The content of biobased carbon is typically assessed by means of carbon-14 dating (also called carbon dating or radiocarbon dating). In addition, in the present invention, the “content of biobased carbon” refers to the molar ratio of biobased carbon to total carbon in the compound or product. The content of biobased carbon may preferably be measured by a method consisting in measuring the decay process of ¹⁴C (carbon-14), in disintegrations per minute per gram of carbon (or 10 dpm/gC), by liquid scintillation counting, preferably in accordance with the standard test method ASTM D6866-16. Said American Standard Test Method ASTM D6866 is considered equivalent to the standard ISO 16620-2. According to said standard ASTM D6866, the test method may preferably use AMS (Accelerator Mass Spectrometry) techniques with ¹³C IRMS (Isotope Ratio Mass Spectrometry) to quantify the biobased content of a given product. Carbon atoms coexist naturally with their stable ¹³C isotopes. The amount and ratio of ¹³C/¹²C is influenced by several factors such as in particular the environment for the natural products. The isotopic fingerprint of a product gives information regarding the origin of the product, in particular the natural or fossil origin. The ¹³C—SNIF-NMR method measures the ¹³C/¹²C ratio of each site of a molecule.

The mean ¹³C isotopic deviation (δ¹³C) is measured by isotope ratio mass spectrometry (IRMS) relative to PDB (pee bee belemnite), the international reference standard.

The present invention relates to a process for preparing para-hydroxycinnamic acid of formula (I)

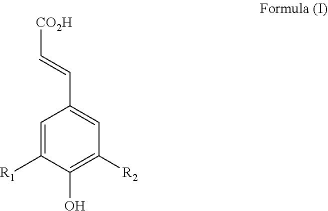

in which R1 and R2 are, identical or different, independently, chosen from the group consisting of hydrogen, linear or branched, optionally substituted, C1-C6 alkyl groups, linear or branched, optionally substituted, C1-C6 alkenyl groups, —CO2R, —OR, in which R is chosen from hydrogen, linear or branched, optionally substituted, C1-C6 alkyl groups, linear or branched, optionally substituted, C1-C6 alkenyl groups,
in which a para-hydroxybenzaldehyde of formula (II) in which R1 and R2 are as described above

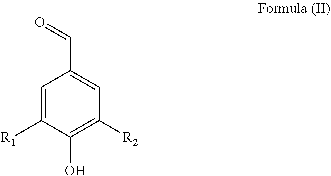

is reacted with malonic acid in the presence of at least one amino acid and acetic acid.
   p-Hydroxycinnamic Acid:

In the context of the present invention, a p-hydroxycinnamic acid refers to a compound of formula (I):

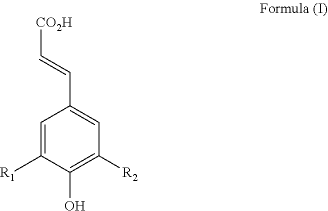

in which R1 and R2 are, identical or different, independently, chosen from the group consisting of hydrogen, linear or branched, optionally substituted, C1-C6 alkyl groups, linear or branched, optionally substituted, C1-C6 alkenyl groups, —CO2R, —OR, in which R is chosen from hydrogen, linear or branched, optionally substituted, C1-C6 alkyl groups, linear or branched, optionally substituted, C1-C6 alkenyl groups.

In the context of the present invention, the carbon-carbon double bond may be trans or cis; preferably, the carbon-carbon double bond is trans. The compound of formula (I) may be a compound of formula (Ia) or (Ib) in which R1, R2 and R are as described above.

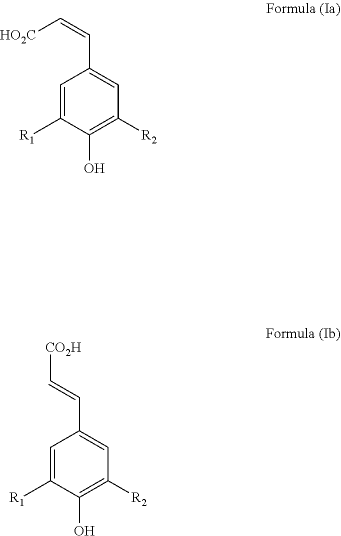

According to a particular aspect, R1 is a hydrogen and R2 is a methoxy or ethoxy group.

The compound of formula (I) may be ferulic acid or 3-(4-hydroxy-3-ethoxyphenyl)prop-2-enoic acid; preferably, the compound of formula (I) is trans-ferulic acid.

According to a particular aspect, the compound of formula (I) obtained in the context of the present invention may be of fossil, biobased, natural or renewable origin.

In the context of the present invention, the content of carbon of biobased origin in the compound of formula (I) is greater than or equal to 50%, preferably greater than or equal to 70%, very preferentially greater than or equal to 90%, for example 97%, 98%, 99%. In the context of the present invention, the content of carbon of biobased origin in the compound of formula (I) is less than or equal to 105%, preferably less than or equal to 103%, very preferentially less than or equal to 100%.

In the context of the present invention, at least 6 carbon atoms in the compound of formula (I) are of biobased origin, preferably at least 7 carbon atoms are of biobased origin, preferably all of the carbon atoms in the compound of formula (I) are of biobased origin.

According to a particular aspect, the compound of formula (I) may have a mean ¹³C isotopic deviation of between −38% and −28% or between −25% and −18%.

p-Hydroxybenzaldehyde:

In the context of the present invention, a p-hydroxycinnamic acid refers to a compound of formula (II):

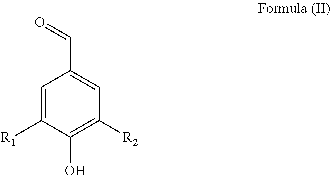

in which R1 and R2 are, identical or different, independently, chosen from the group consisting of hydrogen, linear or branched, optionally substituted, C1-C6 alkyl groups, linear or branched, optionally substituted, C1-C6 alkenyl groups, —CO₂R, —OR, in which R is chosen from hydrogen, linear or branched, optionally substituted, C1-C6 alkyl groups, linear or branched, optionally substituted, C1-C6 alkenyl groups.

According to a particular aspect, R1 is a hydrogen and R2 is a hydrogen or a methoxy or ethoxy group.

The compound of formula (II) may be para-hydroxybenzaldehyde, vanillin or ethylvanillin.

According to a particular aspect, the compound of formula (II) used in the context of the present invention may be of fossil, biobased, natural or renewable origin.

In the context of the present invention, the content of carbon of biobased origin in the compound of formula (II) is greater than or equal to 50%, preferably greater than or equal to 70%, very preferentially greater than or equal to 90%, for example 97%, 98%, 99%. In the context of the present invention, the content of carbon of biobased origin in the compound of formula (II) is less than or equal to 105%, preferably less than or equal to 103%, very preferentially less than or equal to 100%.

In the context of the present invention, at least 6 carbon atoms in the compound of formula (II) are of biobased origin, preferably at least 7 carbon atoms are of biobased origin, preferably all of the carbon atoms in the compound of formula (II) are of biobased origin.

According to a particular aspect, the compound of formula (II) is vanillin that is not of fossil origin.

According to a particular aspect, the compound of formula (II) is vanillin of biobased origin; preferably, the vanillin may have a mean ¹³C isotopic deviation of between −38% and −28% or between −25% and −18%.

### Malonic Acid:

In the context of the present invention, malonic acid refers to a compound of formula (III):

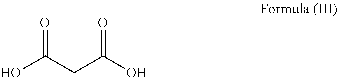

According to a particular aspect, the compound of formula (III) used in the context of the present invention may be of fossil, biobased, natural or renewable origin.

According to a particular aspect, the compound of formula (III) is of fossil origin.

According to a particular aspect, the compound of formula (III) is not of fossil origin.

In the context of the present invention, the content of carbon of biobased origin in the compound of formula (III) is greater than or equal to 50%, preferably greater than or equal to 70%, very preferentially greater than or equal to 90%, for example 97%, 98%, 99%. In the context of the present invention, the content of carbon of biobased origin in the compound of formula (III) is less than or equal to 105%, preferably less than or equal to 103%, very preferentially less than or equal to 100%.

In the context of the present invention, at least 1 carbon atom in the compound of formula (III) is of biobased origin, preferably at least 2 carbon atoms are of biobased origin, preferably all of the carbon atoms in the compound of formula (III) are of biobased origin.

According to a particular aspect, the compound of formula (III) is of biobased origin, and preferably may have a ¹³C isotopic deviation of between −22% c and −17%.

### Amino Acid:

In the context of the present invention, and unless otherwise indicated, the term “amino acid” refers to a compound having both a carboxylic acid function and a primary or secondary amine group. In the context of the present invention, the term “amino acid” preferably refers to proline, glycine, histidine.

### Process:

The present invention relates to a process in which a compound of formula (II) is reacted with malonic acid to form a compound of formula (I).

The process can be described according to the scheme below comprising a first step of condensation between the compound of formula (II) and the malonic acid of formula (III), making it possible to form a diacid intermediate, followed by a step of decarboxylation enabling the formation of the compound of formula (I):

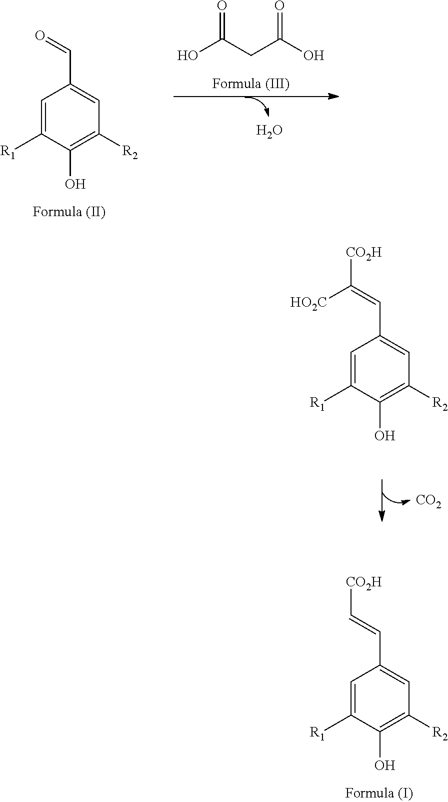

The process of the present invention may be performed continuously or batchwise.

Generally, the process of the present invention is carried out under an inert atmosphere, for example under nitrogen, argon or under oxygen-depleted air.

The process of the present invention is generally performed with stirring.

In general, the process of the present invention is performed at a temperature at which the solvent or the solvent mixture is at reflux, for example at a temperature of between 50° C. and 110° C.

In general, the process of the present invention is performed under reduced pressure or under atmospheric pressure.

In general, the duration of the process is between 2 hours and 24 hours, preferably between 4 h and 12 h.

According to the invention, the process for preparing a compound of formula (I) is carried out in the presence of at least one amino acid and acetic acid.

Generally, the amount of amino acid is between 0.01 equivalent and 1.2 equivalent relative to the amount of compound of formula (II), preferably between 0.05 equivalent and 1.0 equivalent, preferentially between 0.1 equivalent and 0.75 equivalent.

Generally, the amount of malonic acid used is between 1 equivalent and 5 equivalents relative to the compound of formula (II), preferably between 1 equivalent and 2 equivalents, very preferentially between 1 equivalent and 1.5 equivalent, for example 1.2 equivalent.

Generally, the acetic acid is used as solvent. The amount of acetic acid used in the context of the present invention will be able to be adjusted by those skilled in the art; in particular, the amount of acetic acid will be chosen so as to avoid the precipitation of the diacid intermediate.

The use of acetic acid in the context of the present invention has the advantage in particular of stabilizing the malonic acid and the compound of formula (I) obtained, in particular by avoiding the decarboxylation reaction of malonic acid to acetic acid, or of the compound of formula (I), for example to compound of formula (IV).

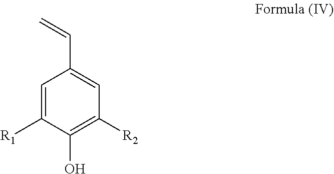

Advantageously, the use of acetic acid as solvent in the context of the process of the present invention allows continuous withdrawal of water produced during the condensation step of the process. The purification of the compound of formula (I) obtained is thus facilitated. By way of illustration, the ferulic acid formed according to the process of the present invention may be crystallized. The process may be performed with continuous withdrawal of water.

In the context of the present invention, the process may be carried out in the presence of a cosolvent. The choice of the cosolvent is not particularly restricted. The cosolvent may be polar, apolar, protic or aprotic. By way of illustration, the cosolvent may be chosen from ethers, alcohols, ketones, aromatic solvents. In general, the cosolvent is preferably chosen from the group consisting of benzene, toluene, DMSO, DMF, dichloromethane, tetrahydrofuran, 2-methyltetrahydrofuran, water, acetone, acetonitrile, 1,4-dioxane or an alcohol of formula R′—OH, or mixtures thereof, where R′ represents a linear or branched alkyl chain comprising from 1 to 6 carbon atoms; preferably, the alcohol is chosen from methanol, ethanol, isopropanol, n-propanol, n-butanol, tert-butanol, 2,2-dimethylpropanol, hexane, pentane and cyclohexane.

Generally, the compound of formula (II), the amino acid and the malonic acid are dissolved in acetic acid. According to another aspect, the compound of formula (II) and the amino acid are dissolved in acetic acid. The malonic acid may then be added directly or dissolved in acetic acid.

The malonic acid dissolved in acetic acid may be added in one go, or over an extended period.

According to a particular aspect, the process for preparing a compound of formula (I) is carried out in the presence of at least one amino acid and a second catalyst preferably chosen from tertiary amines or a pyridine derivative, preferably chosen from pyridine, niacin and nicotinamide.

In the context of the present invention, the amount of second catalyst is between 0.05 equivalent and 1.2 equivalent relative to the amount of compound of formula (II), preferably between 0.1 equivalent and 1.0 equivalent, preferentially between 0.2 equivalent and 0.75 equivalent.

According to a particular aspect, the second catalyst may be introduced in a catalytic amount.

Generally, the amount of second catalyst is between 0.05 equivalent and 0.9 equivalent relative to the amount of compound of formula (II), preferably between 0.1 equivalent and 0.8 equivalent, preferentially between 0.2 equivalent and 0.75 equivalent.

According to a particular aspect, the amino acid and the second catalyst are introduced in catalytic amounts.

Without wishing to be bound by any theory, the inventors have discovered that the joint use of an amino acid and a second catalyst makes it possible to improve the performance of the conversion reaction of the compound of formula (II) to compound of formula (I). The amino acid is in fact suitable for catalyzing the whole process. The second catalyst makes it possible to improve mainly the performance of the decarboxylation step.

The process advantageously makes it possible to improve the conversion of the compound of formula (II) to compound of formula (I). The diacid intermediate compound obtained at the end of the condensation step is effectively decarboxylated to obtain the compound of formula (I).

Overall, since the reaction for forming the compound of formula (I) is more effective, the purification of the compound of formula (I) is facilitated.

The process may be performed with continuous withdrawal of water.

Generally, the compound of formula (II), the amino acid, the second catalyst and the malonic acid are dissolved in acetic acid. According to another aspect, the compound of formula (II), the amino acid and the second catalyst are dissolved in acetic acid. The malonic acid may then be added directly or dissolved in acetic acid.

The compound of formula (I) obtained at the end of the process according to the present invention may be purified by any method known to those skilled in the art, in particular by distillation, purification by chromatography, or crystallization.

Another aspect of the present invention relates to a process for preparing para-hydroxycinnamic acid of formula (I)

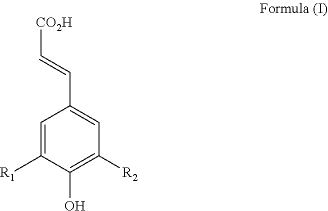

in which R1 and R2 are, identical or different, independently, chosen from the group consisting of hydrogen, linear or branched, optionally substituted, C1-C6 alkyl groups, linear or branched, optionally substituted, C1-C6 alkenyl groups, —CO₂R, —OR, in which R is chosen from hydrogen, linear or branched, optionally substituted, C1-C6 alkyl groups, linear or branched, optionally substituted, C1-C6 alkenyl groups,
in which a para-hydroxybenzaldehyde of formula (II) in which R1 and R2 are as described above

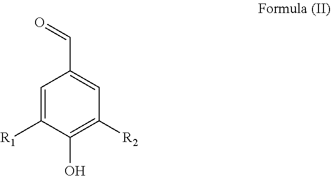

is reacted with malonic acid in the presence of at least one amino acid and a second catalyst, characterized in that the amino acid and the second catalyst are introduced in catalytic amounts.

The process of the present invention is generally performed in a solvent, preferably chosen from the group consisting of DMSO, acetic acid, toluene.

The process of the present invention may be performed continuously or batchwise.

Generally, the process of the present invention is carried out under an inert atmosphere, for example under nitrogen, argon or under oxygen-depleted air.

The process of the present invention is generally performed with stirring.

In general, the process of the present invention is performed at a temperature at which the solvent or the solvent mixture is at reflux, for example at a temperature of between 50° C. and 110° C.

In general, the process of the present invention is performed under reduced pressure or under atmospheric pressure.

In general, the duration of the process is between 2 hours and 24 hours, preferably between 4 h and 12 h.

The process for preparing a compound of formula (I) is carried out in the presence of at least one amino acid in a catalytic amount and a second catalyst, in a catalytic amount, preferably chosen from tertiary amines, niacin and nicotinamide, preferably chosen from pyridine, niacin and nicotinamide.

In the context of this embodiment, the amino acid is introduced in a catalytic amount.

Generally, the amount of amino acid is between 0.01 equivalent and 1.2 equivalent relative to the amount of compound of formula (II), preferably between 0.05 equivalent and 1.0 equivalent, preferentially between 0.1 equivalent and 0.75 equivalent.

In the context of this embodiment, the second catalyst is introduced in a catalytic amount.

Generally, the amount of second catalyst is between 0.05 equivalent and 0.9 equivalent relative to the amount of compound of formula (II), preferably between 0.1 equivalent and 0.8 equivalent, preferentially between 0.2 equivalent and 0.75 equivalent.

The process may be performed with continuous withdrawal of water.

Generally, the compound of formula (II), the amino acid, the second catalyst and the malonic acid are dissolved in a solvent. According to another aspect, the compound of formula (II), the amino acid and the second catalyst are dissolved in a solvent. The malonic acid may then be added directly or dissolved in a solvent.

The compound of formula (I) obtained at the end of the process according to the present invention may be purified by any method known to those skilled in the art, in particular by distillation, purification by chromatography, or crystallization.

Finally, the present invention relates to the use of a compound of formula (I) as synthesis intermediate, preferably for the synthesis of flavorings/aromas. According to a particular aspect, the present invention relates to the use of a compound of formula (I), in which R1 is a hydrogen, R2 is chosen from a methoxy or ethoxy group, for the synthesis of vanillin or ethylvanillin.

### EXAMPLES

### Example 1

Vanillin (6.57 mmol), malonic acid (2 equivalents), and a catalyst, optionally a second catalyst, are mixed in a solvent (6.5 ml) according to table 1. The reaction mixture is stirred under an inert atmosphere at 80° C. for 6 hours.

TABLE 1

| | Catalyst | Solvent | Second catalyst |
|---|---|---|---|
| Example 1.1 | Glycine (0.5 eq.) | Acetic acid | — |
| Example 1.2 | Pyridine (0.5 eq.) | Acetic acid | — |
| Example 1.3 | Glycine (0.25 eq.) | Acetic acid | Pyridine (0.25 eq.) |
| Example 1.4 | Glycine (0.25 eq.) | Acetic acid | Nicotinamide (0.25 eq.) |
| Example 1.5 | Glycine (0.25 eq.) | Acetic acid | Niacin (0.25 eq.) |

The proportions of vanillin, diacid intermediate and ferulic acid are measured by NMR. The yield of ferulic acid is measured by HPLC.

The results are collated in table 2:

TABLE 2

| | | % Diacid | % Ferulic | Yield of |
|---|---|---|---|---|
| | % Vanillin | Intermediate | Acid | ferulic acid |
| Example 1.1 | 9 | 45 | 45 | 36 |
| Example 1.2 | 51 | 4 | 45 | 52 |
| Example 1.3 | 0 | 0 | 100 | 78 |
| Example 1.4 | 11 | 11 | 78 | 69 |
| Example 1.5 | 11 | 12 | 77 | 72 |

### Example 2

Vanillin (6.57 mmol), malonic acid (2 eq.), and a catalyst, optionally a second catalyst, are mixed in a solvent (6.5 ml) according to table 3. The reaction mixture is stirred under an inert atmosphere at 95° C. for 4 h 30.

TABLE 3

| | Catalyst | Solvent |
|---|---|---|
| Example 2.1 | Glycine (0.5 eq.) | DMSO |
| Example 2.2 | Glycine (0.5 eq.) | Acetic acid |

The proportions of vanillin, diacid intermediate, compound of formula (IV) and ferulic acid are measured by NMR.

The results are collated in table 4:

TABLE 4

| | | % Diacid | % | % | % |
|---|---|---|---|---|---|
| | % | Inter- | Ferulic | Formula | Acetic |
| | Vanillin | mediate | Acid | (IV) | acid* |
| Example 2.1 | 8 | 0 | 87 | 4 | 50 |
| Example 2.2 | 15 | 9 | 74 | 2 | N/A |

*The amount of acetic acid is measured as a percentage relative to the amount of VA + Diacid intermediate + Ferulic acid

Example 2.1 shows that the transformation of vanillin into ferulic acid in DMSO in the presence of glycine is possible. However, what is observed under these operating conditions is a greater formation of compound of formula (IV) resulting from the decarboxylation of the ferulic acid. Also observed is the formation of acetic acid resulting from the decarboxylation of the malonic acid. Thus, a large excess of malonic acid and control of the reaction time and/or of the temperature is necessary in order to avoid the formation of compound of formula (IV).

### Example 3

Vanillin (400 mmol), acetic acid (100 ml), glycine (0.1 equivalent) and niacin (0.5 equivalent) are introduced into a round-bottom flask equipped with a Vigreux column and a condenser. The mixture is brought to reflux at 80-82° C. under reduced pressure.

A solution of malonic acid (1.2 equivalent) in acetic acid (300 ml) is added over a period of 2 hours. Solvent is continuously distilled. The distillation is maintained for a period of 2 hours.

After cooling, water is added, and the ferulic acid is crystallized. The solid is filtered off, and washed with water. The ferulic acid is analyzed by NMR and gas-phase chromatography.

The ferulic acid is obtained with a non-optimized yield of 69% and a purity of greater than 97%.

### Example 4

Biobased vanillin (8 carbon atoms of biobased origin, mean ¹³C isotopic deviation=−24% c) is reacted under the conditions of example 3.

Ferulic acid having 8 carbon atoms of biobased origin is obtained.

1. A process for preparing para-hydroxycinnamic acid of formula (I) 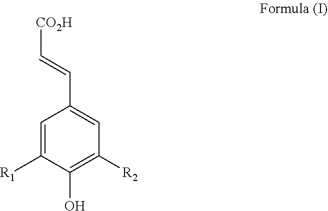
wherein R1 and R2 are, identical or different, independently, chosen from the group consisting of hydrogen, linear or branched, optionally substituted, C1-C6 alkyl groups, linear or branched, optionally substituted, C1-C6 alkenyl groups, —CO₂R, —OR, in which R is chosen from hydrogen, linear or branched, optionally substituted, C1-C6 alkyl groups, linear or branched, optionally substituted, C1-C6 alkenyl groups, the process comprising:
reacting a para-hydroxybenzaldehyde of formula (II) with malonic acid in the presence of at least one of amino acid and acetic acid, 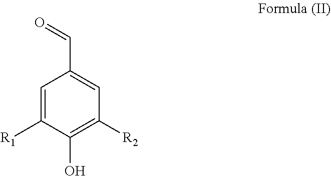
wherein R1 and R2 of formula (II) are as described above.

2. The process as claimed in claim 1, wherein the acetic acid is a solvent.

3. The process as claimed in claim 1, wherein the amount of amino acid is between 0.05 equivalent and 5 equivalents.

4. The process as claimed in claim 1, wherein the amount of amino acid is between 0.01 equivalent and 1.2 equivalent relative to the amount of compound of formula (II).

5. The process as claimed in claim 1, wherein the para-hydroxybenzaldehyde of formula (II) and the malonic acid are reacted in the presence of an amino acid and a second catalyst.

6. The process as claimed in claim 1, wherein a second catalyst is introduced in a catalytic amount.

7. The process as claimed in claim 6, wherein the second catalyst is selected from the group consisting of tertiary amines, niacin, nicotinamide, and combinations thereof.

8. The process as claimed in claim 1, wherein the amino acid is selected from the group consisting of proline, glycine, histidine, and combinations thereof.

9. The process as claimed in claim 1, wherein R1 is a hydrogen and R2 is a methoxy or ethoxy group.

10. The process as claimed in claim 1, wherein the process is conducted at a temperature of between 50° C. and 110° C.

11. The process as claimed in claim 1, wherein the process is carried out in the presence of a cosolvent.

12. The process as claimed in claim 1, wherein the amount of amino acid is between 0.05 equivalent and 1.0 equivalent relative to the amount of compound of formula (II).

13. The process as claimed in claim 1, wherein the amount of amino acid is between 0.1 equivalent and 0.75 equivalent relative to the amount of compound of formula (II).

14. The process as claimed in claim 6, wherein the second catalyst is selected from the group consisting of pyridine, niacin, nicotinamide, and combinations thereof.
